(19) United States
(12) Patent Application Publication
(10) Pub. No.: US 2002/0086997 A1
(43) Pub. Date: Jul. 4, 2002

(54) Method of isolating anhydro-tetrodotoxin

(76) Inventors: Yingzhu Lu, Nanning City (CN); Yan Luo, Nanning City (CN)

Correspondence Address: BIRCH STEWART KOLASCH & BIRCH, PO BOX 747, FALLS CHURCH, VA, 22040-0747, US

(21) Appl. No.: 10/022,521

(22) Filed: Dec. 20, 2001

(30) Foreign Application Priority Data
Dec. 29, 2000 (CN) 00136693.9

(51) Int. Cl. C07D 491/04

(52) U.S. Cl. 544/247

(57) ABSTRACT

The invention relates to a new method of isolating and purifying anhydro-tetrodotoxin (Anh-TTX), comprising a chemical and a high performance liquid chromatography (HPLC) extraction process by which impurities are removed from a solution after tetrodotoxin (TTX) extraction.

### FIELD OF THE INVENTION

The invention relates to a new method of isolating anhydro-tetrodotoxin (Anh-TTX), comprising a chemical and a high performance liquid chromatography (HPLC) extraction process.

### BACKGROUND OF THE INVENTION

Anhydro-tetrodotoxin is a nonprotein neurotoxin with potent activity. Anh-TTX is an amino-perhydroquinozoline compound and a derivative of TTX. TTX is also a nonprotein neurotoxin with potent activity. The TTX molecule consists of a perhydroquinozoline group with a guanidine substituent and six hydroxyl groups. Also being a sodium channel blocker, Anh-TTX possesses a much lower toxicity than does TTX. Its rapid and reversible biological action in blocking a sodium channel allows Anh-TTX to be widely used in the fields of neurobiology, physiology, medical sciences, etc.

The molecular form and structure of Anh-TTX are:

Molecular formula: C₁₁H₁₅O₇N₅

Molecular weight: 301.27

Structure:

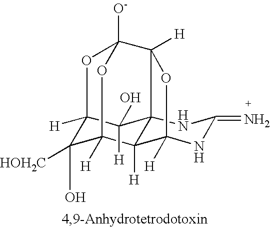

Generally Anh-TTX co-exists with TTX in the same organism. According to Chinese Patent Application Publication No. CN 1206009A, Anh-TTX is found mainly in the ovaries, liver, skin, intestines and stomach of puffer fish, in some organisms found in the ocean and on land, and in bacteria.

The isolation of Anh-TTX from biological tissues or cell cultures involves two major tasks. The first is to remove from Anh-TTX those compounds other than Anh-TTX, TTX, and TTX analogues. Such undesirable compounds include residual amino acids, polypeptides, proteins, and the like. These substances can be removed by, for example, dissolving Anh-TTX in a weak organic acid such as acetic acid, and then precipitating the Anh-TTX from a basic solution such as an ammonia solution. Co-pending application Ser. No. 09/695,711 filed Oct. 25, 2000 describes this process in detail.

The other step is to remove TTX and TTX analogues.

The content of Anh-TTX in organisms is as little as one part per million or even one part in 10 million. This makes it very difficult to extract at high purity. Such difficulties in extraction are seen in methods used by Munetomo Nakamura et al. and Takeshi Yasumoto et al.

In 1984, Munetomo Nakamura et al. obtained 13.4 mg Anh-TTX from puffer fish liver. Their purification can be described as follows [1]:

Pooled livers (1.5 kg) were extracted with 3.9 liters of 0.1% acetic acid by heating in a boiling water bath for 10 min. After filtration, the extract was placed in a separatory funnel and freed of lipids using diethyl ether. An aqueous layer was applied successively to columns of activated charcoal and Bio-Gel P-2 in a manner employed for isolation of TTX from the Japanese ivory shell (Yasumoto et al., Bull. Jap. Soc. Scient. Fish. 47:939 (1981)). One TTX derivative could be separated as it was not retained on the Bio-Gel P-2 column. Further purification of this compound was carried out by gel permeation chromatography on a Toyopearl HW-40 column with 50% methanol, then by HPLC on a TSK-gel G1000PW column (Toyosoda Co.) with 0.1 acetic acid, and finally by HPLC on a cation-exchange column (Hitachi-gel 3011C) with 0.1% acetic acid. The other two derivatives retained on the Bio-Gel P-2 column were eluted with 0.05 N acetic acid. After solvent removal, the residue was chromatographed over a Bio-Rex 70 column by gradient elution with acetic acid (0-0.06 N). The linear gradient elution chromatography of each derivative was repeated over the Hitachi-gel 3011C column. Further purification was performed by HPLC on the TSK-gel G1000PW column with 0.05 N acetic acid. Elution of each derivative from the columns was monitored by a UV monitor (JASCO UVIDEC-100-11) at 230 nm.

Takeshi Yasumoto et al. (1987) extracted 20 mg Anh-TTX from 3.5 kg of newt tissues by the same procedure.

The approach used in these two examples is characterized by complexity and low yield.

### SUMMARY OF THE INVENTION

The present invention resides in a method for extracting Anh-TTX and removing impurities. Anh-TTX purity levels obtained by the invention can be as high as 97% or more. The method for purification of Anh-TTX of this invention comprises the following steps:

a. preparing a sample by dissolving a substance comprising Anh-TTX in a weak acid, preferably in an organic acid;

b. applying the Anh-TTX solution to a chromatography column comprising an alkylsilane (preferably having a C₁₂ to C₂₄alkyl chain) stationary phase and chromatographically separating the sample using a mobile phase comprising an aqueous alkyl sulfonate solution;

c. collecting the portion that contains Anh-TTX;

d. concentrating the collected solution under vacuum, preferably at 40-60° C.;

e. adjusting the pH of the concentrated solution to basic (9-11) with an alkaline solution, preferably aqueous ammonia or a hydroxide solution;

f. precipitating the Anh-TTX from the solution at a temperature between 0-25° C., preferably 2-8° C.;

g. washing the precipitate, preferably with de-ionized water; and

h. vacuum drying and crystallizing Anh-TTX.

The raw material for the present process can be the residual solution resulting from HPLC purification of TTX (See Chinese Patent Application 00132673.2 for details of TTX purification). A similar method for purifying TTX is found in co-pending U.S. patent application Ser. No. 09/818,863, filed Mar. 28, 2001, the disclosure of which is incorporated herein by reference in its entirety. Alternatively, the starting material can be crude toxin extracted from animal tissues, e.g., as described in co-pending application Ser. No. 09/695,711.

TTX and Anh-TTX often co-exist in a crude toxin solution, so both can be purified in a single HPLC run under conditions described below. TTX and Anh-TTX are isolated at different retention times, with Anh-TTX being retained on the column longer than TTX.

### BRIEF DESCRIPTION OF THE DRAWINGS

FIG. 1: Extraction flow chart of this invention.

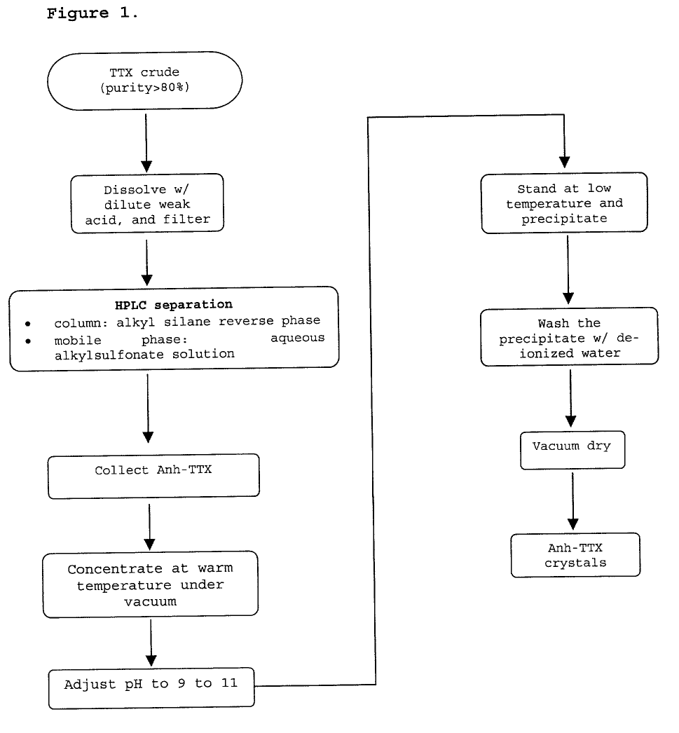

FIG. 2: HPLC Profile of Anhydro-Tetrodotoxin.

FIG. 3: ¹H-NMR Spectrum of Anhydro-Tetrodotoxin.

FIG. 4: ¹H-NMR Spectrum of Anhydro-Tetrodotoxin.

FIG. 5: ¹H-NMR Spectrum of Anhydro-Tetrodotoxin.

FIG. 6: ¹H-¹H Cosy Spectrum of Anhydro-Tetrodotoxin.

FIG. 7: ¹³C-NMR Spectrum of Anhydro-Tetrodotoxin.

FIG. 8: The HRMS Profile of Anhydro-Tetrodotoxin.

FIG. 9: Infrared Absorption Spectrum of Anhydro-Tetrodotoxin.

FIG. 10: HPLC Profile of Anhydro-Tetrodotoxin (Example 3).

### DETAILED DESCRIPTION OF THE INVENTION

The following general procedures should be followed to extract Anh-TTX:

First, prepare a sample by dissolving a substance comprising Anh-TTX in a weak organic acid, such as formic acid, acetic acid, or propionic acid, or in an inorganic acid, such as phosphoric acid, sulfuric acid, or hydrochloric acid. The substance comprising Anh-TTX can be 100-1000 mg crude TTX, for example, prepared as described in co-pending Application Ser. No. 09/695,711, hereby incorporated by reference in its entirety.

Next, apply the Anh-TTX solution to a chromatography column comprising an octyldecylsilane (ODS) stationary phase and chromatographically separating the sample using a mobile phase comprising an aqueous alkyl sulfonate solution. Reversed phase chromatography (RPC) is used in separating TTX and Anh-TTX, and ODS column is preferred.

A RESOURCE RPC 3 mL column produced by Amersham Pharmacia Biotech can also be used to isolate TTX and Anh-TTX. The resource RPL matrix is monodisperse underivatized polystyrene/divinyl benzene beads.

C₃ to C₁₃ alkyl sulfonates may be selected as ion-pair reagents for preparing the mobile phase. The range of concentration of alkyl sulfonate is between 0.001-0.05 mol/L, preferably 0.002-0.02 mol/L.

Then collect the portions containing Anh-TTX and TTX.

Concentrate the collected Anh-TTX portion, preferably at 40-60° C. under vacuum.

Perform chemical treatment to the concentrated solution: Adjust the pH of the solution to a value between 9-11 with ammonia or other basic solution (1-10%). Allow the solution to stand at 2-40° C. for a period of time so that impurities will decompose or convert into Anh-TTX by chemical reactions and the Anh-TTX substance will precipitate.

Obtain Anh-TTX by washing the Anh-TTX precipitate three times with de-ionized water, and then vacuum drying the precipitate to obtain Anh-TTX crystals of high purity. To obtain purity levels of 97% or higher, the Anh-TTX can be recrystallized by redissolving the crystals in weak acid, adjusting the pH to 9 to 11, allowing precipitation and washing the crystals, and then vacuum drying the precipitate to obtain Anh-TTX crystals.

### EXAMPLES

The following examples serve to illustrate the invention, but are in no way intended to limit the invention.

### Example 1

1. Collection of Anh-TTX portion during purification of TTX by HPLC:

1) 100 mg of crude TTX, having a purity of 80.5% and obtained from puffer fish ovaries by the method described in Co-pending application Ser. No. 09/695,711, was taken and dissolved in 2.5 mL 5% acetic acid, then filtered and separated by HPLC.

2) HPLC conditions:

a. Extraction column: ODS (5 μm), 300×19 mm.

b. Mobile phase: 0.01 M sodium heptane sulfonate, flow rate 10 mL/min.

c. Quantitative injection: 200 pL per injection.

d. UV detector: detection wavelength 201 nm.

3) Collecting the portions of TTX and Anh-TTX:

a. The retention time of TTX was 20.5-27.0 min.

b. The retention time of Anh-TTX was 36.5-41.0 min.

c. The volume of the collected Anh-TTX portion was 250 mL.

2. Concentration of the collected Anh-TTX portion:

The collected Anh-TTX solution was concentrated to a volume of 15 mL by rotatary evaporation at 0.005 MPa at 45° C.

3. Chemical treatment of the concentrated solution:

The pH of the concentrated solution was adjusted to 910 with 5% ammonia, then allowed to stand at 4° C. for 2 days to precipitate Anh-TTX.

4. Obtaining Anh-TTX:

The precipitate was washed 3 times with de-ionized water, then desiccated under vacuum; 10 mg Anh-TTX was obtained.

### Example 2

1.

1) 430 mg of crude TTX, having a purity of 82.6% and obtained from puffer fish ovaries by the method described in Co-pending application Ser. No. 09/695,711, was dissolved in 4 mL 8% acetic acid, then filtered and separated by HPLC.

2) HPLC conditions:

a. Extraction column: ODS (5 μm), 300×19 mm.

b. Mobile phase: 0.01 M sodium heptane sulfonate, flow rate 5 mL/min.

c. Quantitative injection: 200 μL per injection.

d. UV detector: detection wavelength 201 nm.

3) Collecting the portions of TTX and Anh-TTX:

a. The retention time of TTX was 20-28 min.

b. The retention time of Anh-TTX was 34-39 min.

c. The volume of the collected Anh-TTX portion was 1000 mL.

2. Concentration of the collected Anh-TTX portion:

The collected Anh-TTX solution was concentrated to a volume of 40 mL by rotatary evaporation at 0.005 MPa at 50° C.

3. Chemical treatment of the concentrated solution:

The pH of the concentrated solution was adjusted to 910 with 5% ammonia, then allowed to stand at room temperature for 1 day, then at 4° C. for 2 days to precipitate Anh-TTX.

4. Obtaining Anh-TTX:

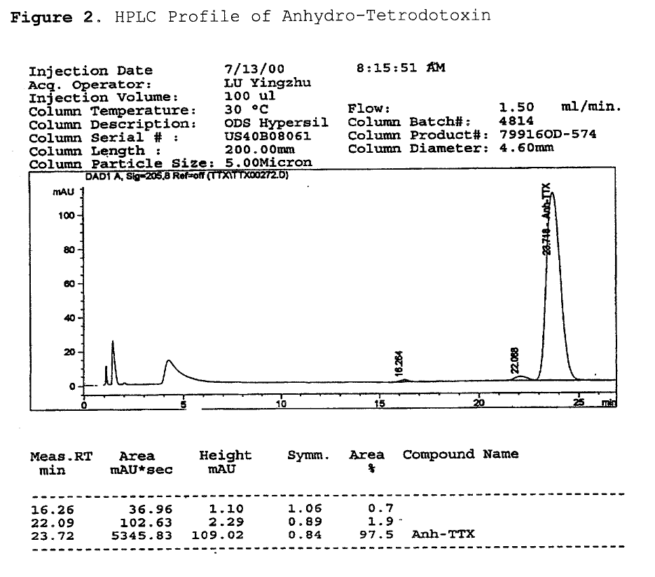

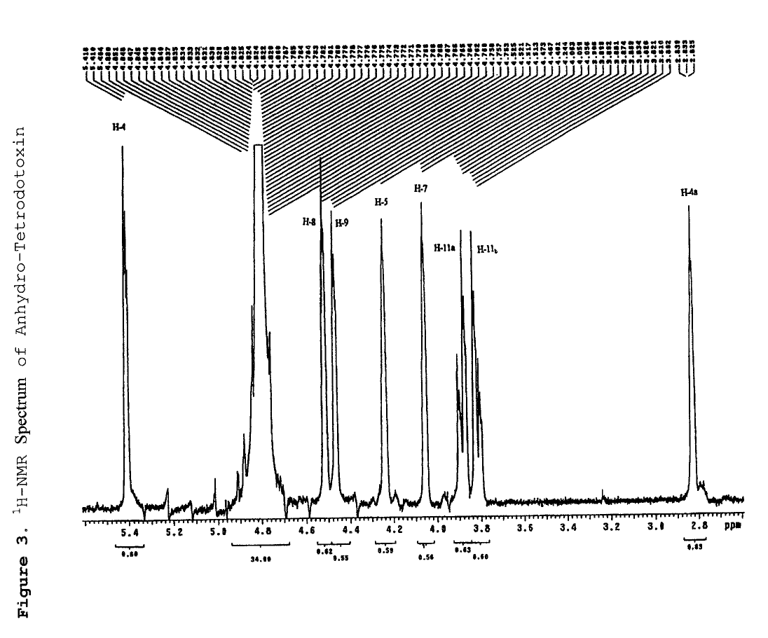

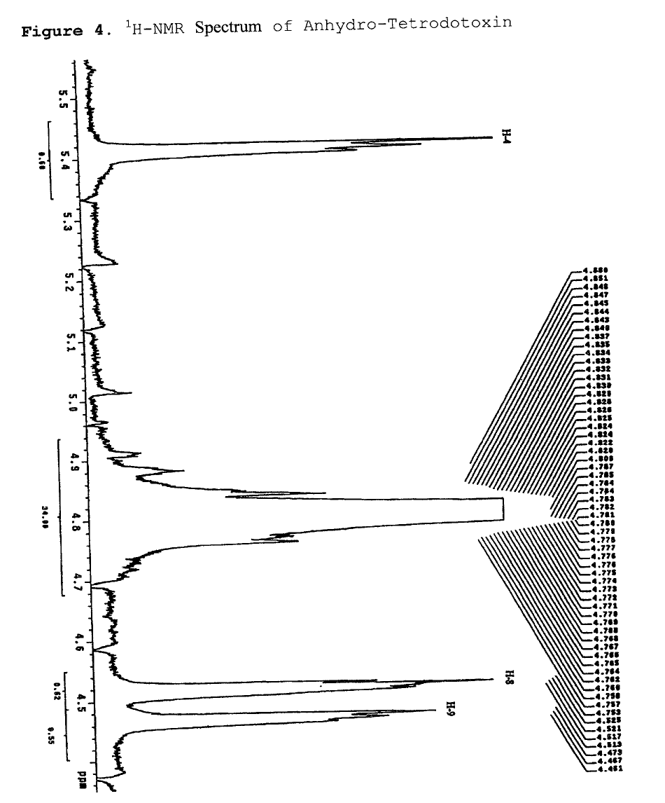

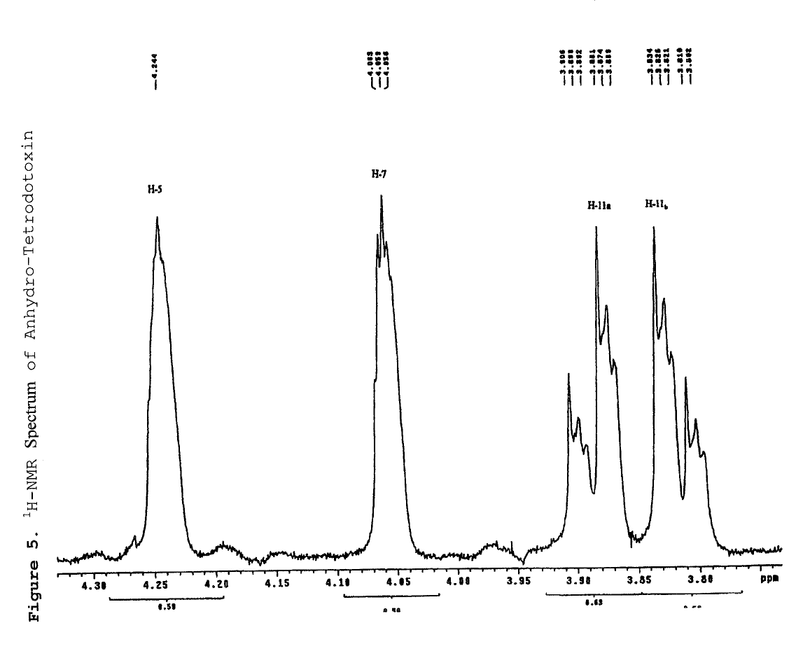

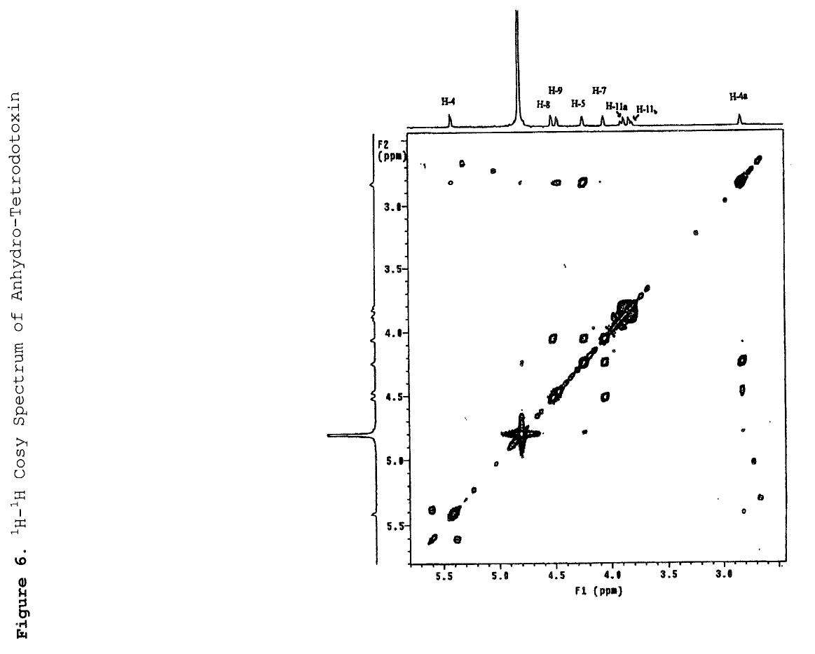

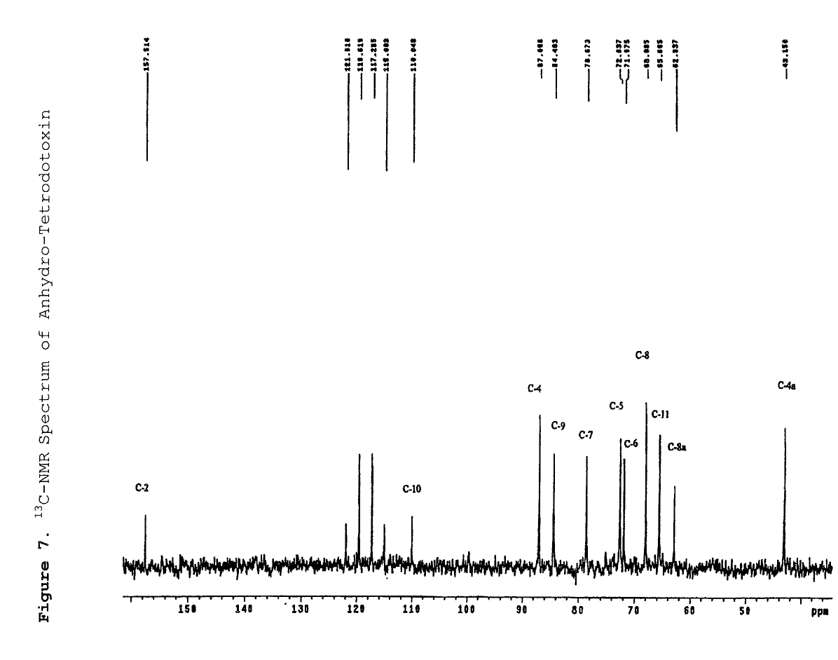

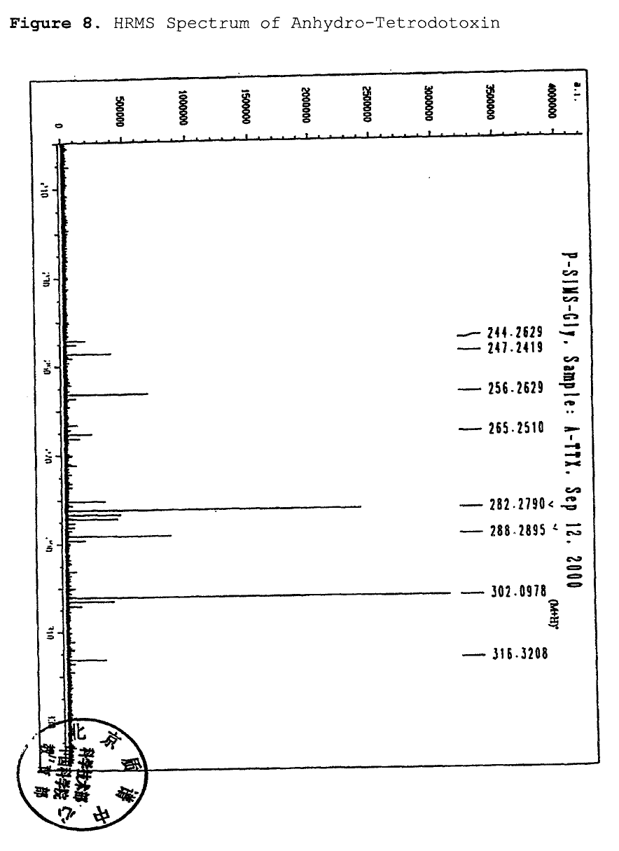

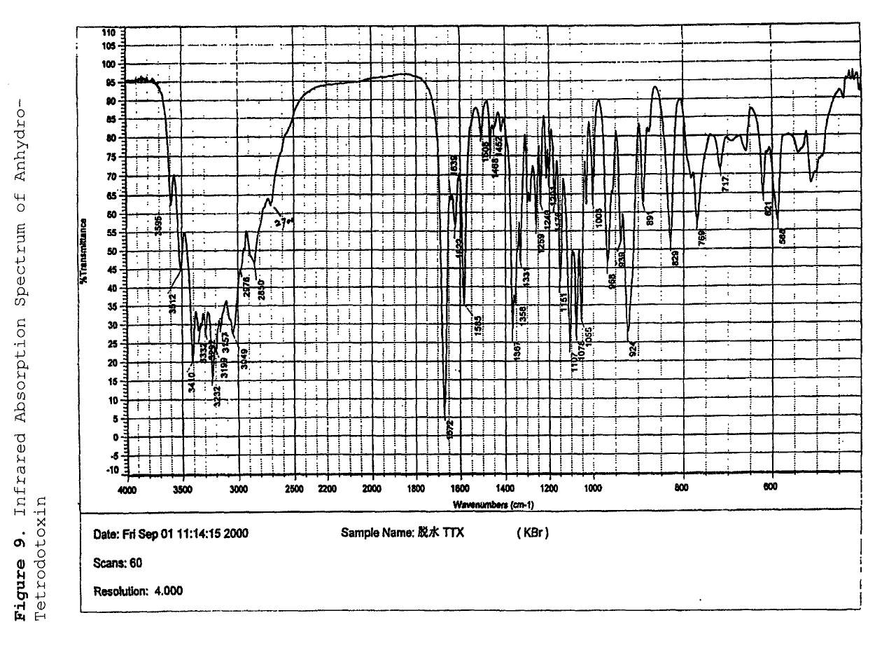

The precipitate was washed 3 times with de-ionized water, then desiccated under vacuum. 30 mg Anh-TTX was obtained with a purity of 97.5%. Part of the substance was sent to China Academy of Medical Sciences, the Pharmaceutical Institute of China Union Medical University and National Analytical Center for Pharmaceuticals and Metabolites of China for structure verification (FIGS. 2-9).

### Example 3

1.

1) 1000 mg of crude TTX, having a purity of 82.0% and obtained from puffer fish ovaries by the method described in Co-pending application Ser. No. 09/695,711, was dissolved in 10 mL 10% acetic acid, then filtered and separated by HPLC.

2) HPLC conditions:

a.Extraction column: ODS (5 μm), 300×19 mm.

b.Mobile phase: 0.01 M sodium heptane sulfonate, flow rate 10 mL/min.

c.Quantitative injection: 200 μL per injection.

d.UV detector: detection wavelength 201 nm.

3) Collecting the portions of TTX and Anh-TTX:

a. The retention time of TTX was 20-28 min.

b. The retention time of Anh-TTX was 34-39 min.

c. The volume of the collected Anh-TTX portion was 2000 mL.

2. Concentration of the collected Anh-TTX portion:

The collected Anh-TTX solution was concentrated to a volume of 60 mL by rotatary evaporation at 0.005 MPa at 50° C.

3. Chemical treatment of the concentrated solution:

The pH of the concentrated solution was adjusted to 9-10 with 1M sodium hydroxide, then allowed to stand at room temperature for 1 day, then at 4° C. for 3 days to precipitate Anh-TTX.

4. Obtaining Anh-TTX:

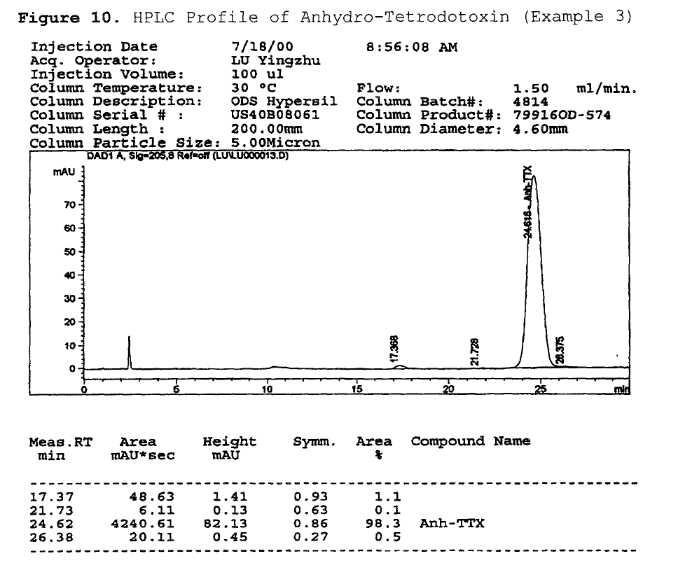

The precipitate was dissolved in 25 mL 0.5% acetic acid, then its pH was adjusted to 9-10 with 10% ammonia to precipitate again. Successively, the precipitate was washed 3 times with de-ionized water, then desiccated under vacuum. 65 mg Anh-TTX was obtained with a purity of 98.3% (FIG. 10).

### REFERENCES

Various articles of the scientific and patent literature are cited throughout this document. Each such article is hereby incorporated by reference in its entirety for all purposes by such citation.

[1] Munetomo Nakamura and Takeshi Yasumoto, “Tetrodotoxin derivatives in puffer fish,” Toxicon 23, 271-276 (1985).

[2] Takeshi YasumotoMari Yotsu and Michio Murata, “New Tetrodotoxin Analogues from the Newt cynops ensicauda,” J. Am. Chem. Soc. 110, 2344-2345 (1988).

[3] Zhiqing Huang et al., “A Method of Extracting AnhydroTetrodotoxin”, Chinese Patent Application Publication No. CN 1206009A (1999).

We claim:

1. A method of isolating anhydro-tetrodotoxin comprising the steps of:
a) dissolving a substance comprising anhydro-tetrodotoxin in a weak organic acid or in an inorganic acid to obtain an anhydro-tetrodotoxin solution;
b) applying the anhydro-tetrodotoxin solution to a chromatography column comprising an alkylsilane stationary phase and chromatographically separating the sample using a mobile phase comprising an aqueous alkyl sulfonate solution;
c) collecting the fraction containing anhydro-tetrodotoxin;
d) adjusting the pH of the anhydro-tetrodotoxin fraction to alkaline;
e) precipitating the anhydro-tetrodotoxin;
f) washing and drying the precipitate to obtain anhydro-tetrodotoxin; and optionally
g) re-crystallizing anhydro-tetrodotoxin from the precipitate.

2. The method of claim 1, wherein the substance comprising anhydro-tetrodotoxin in step a) is 100-1000 mg crude TTX.

3. The method of claim 1, wherein the weak organic acid in step a) is selected from the group consisting of: formic acid, acetic acid, and propionic acid.

4. The method of claim 1, wherein the inorganic acid in step a) is selected from the group consisting of: phosphoric acid, sulfuric acid, and hydrochloric acid.

5. The method of claim 1, wherein the chromatography column in step b) comprises an octyldecylsilane stationary phase.

6. The method of claim 1, wherein the aqueous alkyl sulfonate solution in step b) is an aqueous sodium heptane sulfonate solution.

7. The method of claim 1, wherein step c) is performed at 40-60° C. under vacuum.

8. The method of claim 1, wherein step d) is performed using aqueous ammonia or a hydroxide solution.

9. The method of claim 1, wherein in step d), the pH is adjusted to 9-11.

10. The method of claim 1, wherein step e) is performed at a temperature from 0-25° C.

11. The method of claim 1, wherein step e) is performed at a temperature from 2-8° C.

12. The method of claim 1, wherein step e) is performed using de-ionized water.

13. Anhydro-tetrodotoxin prepared by the method of claim 1.
